(19) United States
(12) Patent Application Publication
(10) Pub. No.: US 2014/0147525 A1
(43) Pub. Date: May 29, 2014

(54) Two Component Systems For Delivering Stabilized Ascorbic Acid

(71) Applicant: JOHNSON & JOHNSON COSUMER COMPANIES, INC., Skillman, NJ (US)

(72) Inventors: Leonardo de Paula, Sao Jose Dos Campos (BR); Filomena Macedo, Hillsborough, NJ (US); Cristina Vendruscolo, Sao Jose dos Campos (BR); Cinthia Zanatta, Sao Jose dos Campos (BR)

(73) Assignee: JOHNSON & JOHNSON CONSUMER COMPANIES, INC., Skillman, NJ (US)

(21) Appl. No.: 13/685,090

(22) Filed: Nov. 26, 2012

(51) Int. Cl. A61K 8/67; A61K 8/97; A61Q 19/00

(52) U.S. Cl. CPC A61K 8/676; A61Q 19/00; A61K 8/97
USPC 424/736; 514/474

(57) ABSTRACT

Stable, topical compositions comprising high levels of ascorbic acid are provided, which comprise an anhydrous component containing at least 15 weight % ascorbic acid and an oil-in-water emulsion component combined at the point of use. Such compositions provide increased permeation of ascorbic acid.

### FIELD OF INVENTION

The present invention relates to a two component system for delivering high amounts of stable ascorbic acid to the skin. In addition to maintaining the stability of ascorbic acid, the system provides enhanced permeation of the ascorbic acid into the skin.

### BACKGROUND OF THE INVENTION

Vitamin C, known by its common name of ascorbic acid, is a naturally occurring organic compound having anti-oxidant properties that benefit the skin. It helps neutralize the damaging effects of free radicals and may help stimulate the growth of collagen that is important in maintaining healthy skin elasticity and texture. Wrinkle reduction is one of its primary uses. Vitamin C can also treat or prevent other conditions cause by UV-A and UV-B radiation such as UV-B radiation-induced erythema, photoaging of the skin, skin cancer, scar tissue formation, etc.

Vitamin C in the form of L-ascorbic acid is the chemical form of ascorbic acid that is reported to be most effectively utilized by the body but water-based formulations containing ascorbic acid are typically not stable. Although ascorbic acid is readily soluble in water, rapid oxidation occurs in aqueous media. Eliminating water from the formulation cures this problem.

U.S. Pat. No. 4,818,521 (Tamabuchi) relates to a premix of L-ascorbic acid with an oil that may be a silicone oil, which is then combined with an emulsion. Tamabuchi also describes in the Background of the Invention section a so-called two-pack type cosmetic wherein Vitamin C powder and other ingredients are separately packaged in different containers with mixing just prior to use of the cosmetic. However, the mixing procedure and expensive packaging were said to be drawbacks of the two part system.

U.S. Pat. No. 5,853,741 (Znaiden et al.) relates to ascorbic acid solubilized by a crosslinked non-emulsifying siloxane elastomer in a carrier medium of a volatile siloxane.

Despite the teachings of Tambuchi and Znaiden et al., the solubility of ascorbic acid in non-aqueous media is known to be relatively poor. In addition, permeation of ascorbic into skin is known to decrease when delivered from silicone-based vehicles.

Conventional non-aqueous ascorbic acid products that contain either waxes or combinations of waxes and oils to promote stability of the suspensions also leave an oily or waxy residue on the skin after use that is unappealing and unaesthetic.

Applicants have discovered a new way to stabilize ascorbic acid-containing compositions that also deliver high amounts of ascorbic acid to the skin when topically administered. The compositions provide enhanced penetration, thereby delivering skin benefits of lightening, improving signs of aging, and reducing inflammation.

### SUMMARY OF THE INVENTION

The invention provides a two-part composition for topical application of ascorbic acid to skin comprising: a) an anhydrous component consisting essentially of greater than 15 weight % ascorbic acid suspended in a silicone vehicle; and b) an oil-in-water emulsion component comprising a permeation enhancer; wherein said anhydrous component and said oil-in-water emulsion component are separated until topically applied to said skin.

The invention further provides a method of administering ascorbic acid to skin, which comprises in sequence: (1) mixing a) an anhydrous component consisting essentially of greater than 15 weight % ascorbic acid suspended in a silicone vehicle with b) an oil-in-water emulsion component comprising a permeation enhancer to form a mixture; and (2) topically applying said mixture to said skin within two minutes of said mixing.

### DETAILED DESCRIPTION OF THE INVENTION

Applicants have discovered unexpectedly that topical delivery of high levels of ascorbic acid is possible from a two component composition that also provides increased stability of the ascorbic acid and increased penetration of the ascorbic acid into the skin. This system may be used in skin care compositions, and in methods of using such compositions for, but not limited to, treating skin in need of treatment for skin lightening, wrinkle reduction, improving signs of aging, and reducing inflammation.

Unless defined otherwise, all technical and scientific terms used herein have the meaning commonly understood by one of ordinary skill in the art to which the invention pertains. All publications, patent applications, patents, and other references mentioned herein are incorporated by reference. Unless otherwise indicated, a percentage refers to a percentage by weight (i.e., % (W/W)).

As used herein, “cosmetically-acceptable” means suitable for use in topical contact with tissues (e.g., the skin) without undue toxicity, incompatibility, instability, irritation, allergic response, or the like. This term is not intended to limit the composition it describes as for use solely as a cosmetic (e.g., the composition may be used as a pharmaceutical).

As used herein, the terminology “safe and effective amount” means an amount sufficient to provide a desired benefit at a desired level, but low enough to avoid serious side effects.

As used herein, the terminology “treating” or “treatment” means alleviation or elimination of symptoms, cure, prevention, or inhibition of a human condition or disease, specifically of the skin.

As used herein, “skin in need of treatment for signs of aging” means a skin that is, but not limited to, sagging, loose, lax, rough, wrinkly, thinned, or uneven. Improving the signs of aging includes improving the firmness of the skin, improving the texture of the skin, improving the appearance of wrinkles in skin, improving the skin tone (including lightning specific age spots or the skin in general) or the treatment of external aggressions in skin.

As used herein, “improving the firmness of skin” means the enhancing of the firmness or elasticity of the skin, preventing the loss of firmness or elasticity of skin, or preventing or treating sagging, lax and loose skin. The firmness or elasticity of the skin can be measured by use of a cutometer. See Handbook of Non-Invasive Methods and the Skin, eds. J. Serup, G. Jemec & G. Grove, Chapter 66.1 (2006). The loss of skin elasticity or firmness may be a result of a number of factors, including but not limited to aging, environmental damage, or the result of an application of a cosmetic to the skin.

As used herein, “improving the texture of skin” means the smoothing of the surface of the skin to remove either bumps or crevasses on the skin surface.

As used herein, “improving the appearance of wrinkles in skin” means preventing, retarding, arresting, or reversing the process of wrinkle and fine line formation in skin.

As used herein, “treatment of external aggressions in skin” means the reduction or prevention of the damage from external aggressions in skin. Examples of external aggressions include, but are not limited to, damage to the skin from the use of cleansers (e.g., topical cleansers containing surfactants), make-up, shaving as well as environmental damage such as from UV light (e.g., sundamage from sunlight or damage from non-natural sources such as UV lamps and solar simulators), ozone, exhaust, pollution, chlorine and chlorine containing compounds, and cigarette smoke. Effects of external aggressions on the skin include, but are not limited to, oxidative and/or nitrosative damage to and modifications on lipids, carbohydrates, peptides, proteins, nucleic acids, and vitamins. Effects of external aggressions on the skin also include, but are not limited to, loss of cell viability, loss or alteration of cell functions, and changes in gene and/or protein expression.

As used herein, “improving the skin tone” means the lightening of the appearance of the skin (e.g., lightening pigmented marks or lesions, reducing skin sallowness, and/or evening the color of the skin).

As used herein, “skin in need of treatment for skin inflammation” means a skin exhibiting redness or erythema, edema, or being reactive or sensitive to external elements. External elements include, but are not limited to, sun rays (UV, visible, IR), microorganisms, atmospheric pollutants such as ozone, exhaust pollutants, chlorine and chlorine generating compounds, cigarette smoke, cold temperature, heat. Inflammatory disorders and related conditions which may be treated or prevented by use of the compositions of this invention include, but are not limited to the following: arthritis, bronchitis, contact dermatitis, atophic dermatitis, psoriasis, seborrheic dermatitis, eczema, allergic dermatitis, polymorphous light eruptions, inflammatory dermatoses, folliculitis, alopecia, poison ivy, insect bites, acne inflammation, irritation induced by extrinsic factors including, but not limited to, chemicals, trauma, pollutants (such as cigarette smoke) and sun exposure, secondary conditions resulting from inflammation including but not limited to xerosis, hyperkeratosis, pruritus, postinflammatory hyperpigmentation, scarring and the like. Preferably, the inflammatory disorders and related conditions which may be treated or prevented using the methods of the invention are arthritis, inflammatory dermatoses, contact dermatitis, allergic dermatitis, atopic dermatitis, polymorphous light eruptions, irritation, including erythema induced by extrinsic factors, acne inflammation, psoriasis, seborrheic dermatitis, eczema, poison ivy, insect bites, folliculitus, alopecia, and secondary conditions and the like.

As used herein, the term “lightening the skin” refers generally to lightening, brightening, whitening, and/or evening of the skin tone, skin color, and/or shade of skin, and/or to the reduction in sallowness, and/or to the lightening and/or fading of hyperpigmented marks and/or lesions including, but not limited to, pigmented spots, melanin spots, age spots, sun spots, senile lentigos, freckles, lentigos simplex, pigmented solar keratosis, seborrhoeic keratosis, melasma, acne marks, post-inflammatory hyperpigmentation, lentigines, ephelides, combinations of two or more thereof and the like. In certain embodiments, “lightening the skin” also refers to increased skin radiance, glow, translucency and/or luminescence and/or obtaining a more radiant, glowing, translucent or luminous skin tone appearance or a less yellow or sallow skin tone. In certain preferred embodiments, “lightening the skin” refers to lightening and evening the skin tone, increasing skin radiance and/or lightening age spots.

In certain other preferred embodiments, the present invention is directed to compositions and methods for use on skin in need of skin lightening treatment selected from the group consisting of age spots, freckles, marks left after acne, and combinations of two or more thereof.

As used herein, unless otherwise specified, all percentages of ingredients in compositions are weight percent of active/solids ingredient based on the total weight of composition.

As used herein, “cosmetically/ dermatologically acceptable” means that the ingredients which the term describes are suitable for use in contact with tissues (e.g., the skin or hair) without undue toxicity, incompatibility, instability, irritation, allergic response, and the like.

According to the invention, the stability and permeation of the ascorbic acid is increased.

For example, application of the composition of the invention provides for permeation of two or three or five or seven times the amount of ascorbic acid into the skin compared with application of only an anhydrous component containing ascorbic acid.

The pH of the composition may be below about 4, or below about 3.5.

### Anhydrous Component

The two-part composition comprises an anhydrous component consisting essentially of ascorbic acid suspended in a silicone vehicle. The anhydrous component may consist of ascorbic acid suspended in a silicone vehicle.

The amount of ascorbic acid in the silicone vehicle is greater than 15 weight % of the total weight of anhydrous component. Preferably, the amount of ascorbic acid in the silicone vehicle is greater than 20 weight %, or greater than 30 weight %, of the total weight of anhydrous component. The amount of ascorbic acid may be greater than 35 weight % of the total weight of the anhydrous component.

As used herein, “anhydrous” means containing less than about 0.25 weight % water. In a preferred embodiment, the anhydrous component contains less than about 0.1 weight % water. In yet a further preferred embodiment, the anhydrous component is completely free of water.

Ascorbic acid is available from several commercial sources including DSM Nutritional Products, Orient Stars LCC, and Universal Presery-A-Chem. Inc.

The silicone vehicle of the anhydrous component may contain a volatile or non-volatile siloxane crosspolymer and a volatile or non-volatile siloxane. For example, the silicone vehicle may comprise a non-emulsifying non-volatile siloxane crosspolymer in dimethicone.

Preferred siloxane crosspolymers include for example Dimethicone/Cetearyl Dimethicone Crosspolymer (VELVESIL DM), Cyclopentasiloxane/C30-45 Alkyl Cetearyl Dimethicone Crosspolymer (VELVESIL 125), Cyclopentasiloxane/Boron Nitride/Caprylyl methicone/C30-45 Alkyl Cetearyl Dimethicone Crosspolymer (VELVESIL FX), Cyclopentasiloxane/C30-45 Alkyl Cetearyl Dimethicone Crosspolymer/PEG/PPG-20/23/Dimethicone (VELVESIL Plus), and Caprylyl methicone/C30-45 Alkyl Cetearyl Dimethicone Crosspolymer (VELVESIL 034). All VELVESIL products are commercially available from Momentive Performance Materials. Other preferred non-volatile siloxane crosspolymers include Dimethicone/Cetearyl Dimethicone Crosspolymer (BOTANISIL GB-25 commercially available from Botanigenics, Inc), Dimethicone Cetearyl/Dimethicone Crosspolymer/PEG/PPG-20/23/Dimethicone (Y-17483 commercially available from Momentive Performance Materials), and Dimethicone/Boron Nitride/Caprylyl Methicone/Cetearyl Dimethicone Crosspolymer/PEG/PPG-20-23/Dimethicone) (Y-17583 commercially available from Momentive Performance Materials.

Other hydrophobic ingredients such as other siloxanes, silanes, oils, waxes and the like known in the art may be included in the anhydrous component.

For example, the anhydrous component may include an isododecane crosspolymer.

The anhydrous component may include a siloxane such as Caprylyl Methicone. Caprylyl Methicone is commercially available as SILSOFT 034 from Momentive Performance Materials.

The siloxane may be a non-volatile siloxane such as dimethicone, for example DC 200/50 CS (commercially available from Dow Corning).

Volatile siloxanes may also be used in the anhydrous component. Examples include cyclomethicone, cyclopentasiloxane, cyclohexasiloxane, triphenyl, trimethicone and the like.

The amount of silicone vehicle in the anhydrous component is typically from about 50% to about 95%, preferably from about 60% to about 90%, more preferably from about 65% to about 85% by weight of the anhydrous component.

The proportion of anhydrous component in the final composition may be varied, but it is generally suitable to provide sufficient separation of the water phase particles in the emulsion component, as well as spreadability and pleasant skin-feel. For example, the anhydrous component may comprise about 40 to about 60% by weight or 50% by weight of the final composition, which is the mixture of the anhydrous component and the emulsion component.

### Emulsion Component

The composition also comprises an oil-in-water emulsion component.

The emulsion component may be a “single” oil-in-water emulsion (i.e., a single oil phase dispersed in a single water phase). However, multiphase emulsions, such as oil-in-water-in-oil types or water-in oil-in water types, are also useful in the subject invention.

In general, such single or multiphase emulsions contain water, emollients, and emulsifiers as known in the art.

For example, ingredients suitable for use in the water phase of the oil-in-water emulsion include dissolved salts such as sodium chloride, water soluble surfactants, water-soluble preservatives and dyes, chelating agents (e.g., amino acids such as glycine, edta, citrate, and the like), pH adjusters and buffers (e.g., citric acid, sodium hydroxide, bicarabonate and the like), water-soluble biologically active compounds, glycerin, glycols, and the like.

In certain embodiments of the invention, the amount of water phase in the emulsion component is from 30% to about 90%, preferably from about 40% to about 85%, more preferably from about 50% to about 80%, and most preferably from about 50% to about 75% by weight of the emulsion component. It has been found that relatively high levels of water in the emulsion component result in increased permeation of ascorbic acid into the skin.

Ingredients suitable for use in the oil phase of the oil-in-water emulsion include fatty alcohols for example arachidyl alcohol; behenyl alcohol; caprylic alcohol; cetearyl alcohol; cetyl alcohol; lauryl alcohol; myristyl alcohol; oleyl alcohol; olive alcohol; stearyl alcohol and tridecyl alcohol.

Emollients include for example:

(1) esters such as dicaprylyl carbonate; isononyl isononanoate; diisopropyl adipate, diisopropyl sebacate; isostearyl neopentanoate C12-C15 alkyl benzoate; caprylic/capric glycerides; neopentyl glycol diheptanoate; PPG-3 benzyl ether ethylhexanoate; cetyl palmitate; tribehenin; cetearyl olivate;

(2) silicones and siloxanes such as caprylyl methicone, dimethicone, cyclomethicone; cyclopentasiloxane, cyclohexasiloxane, triphenyl trimethicone;

(3) waxes such as beeswax, candelilla wax, ozoquerita wax;

(4) oils and butters such as lavandula angustifolia (lavender) oil; macadamia oil; sunflower seed oil; sweet almond oil; Vitis vinifera seed oil; oenothera biennis (evening primrose) oil; olive oil; melaleuca oil; Chamomilla recutita (Matricaria) oil; Cinnamomum zeylanicum leaf oil.

Surfactants include for example:

(1) non-ionic surfactants such as: steareth-20, cetareth-20, steareth-2, polysorbate 60, sorbitan oleate, polysorbate 80, cetearyl alcohol/cetearyl glucoside, arachidyl alcohol/behenyl alcohol/arachidyl glucoside, C14-C22 alcohol/C12-C20 alkyl glucoside; inulin lauryl carbamate; coco glucoside/glyceryl oleate/cetearyl glucoside; sorbitan olivate; cetearyl olivate/sorbitan olivate; isostearyl glucoside; polyglyceryl-3 dicitrate/stearate.

(2) anionic surfactants such as: sulfosuccinates and sulfosuccinamates (Disodium Cetearyl Sulfosuccinate); Potassium Cetyl Phosphate.

(3) cationic surfactants as distearyldimonium chloride; behentrimonium methosulfate/cetyl alcohol/butylene glycol.

The emulsion component contains a permeation enhancer. Permeation enhancers include for example liquid fats and oils such as avocado oil, camellia oil, turtle oil, macadamia nut oil, corn oil, mink oil, olive oil, rapeseed oil, egg yolk oil, sesame oil, persic oil, wheat germ oil, sasanqua oil, castor oil, linseed oil, safflower oil, cottonseed oil, perilla oil, soybean oil, peanut oil, tea oil, kaya oil, rice bran oil, Chinese wood oil, Japanese wood oil (Japanese tung oil), jojoba oil, germ oil, glycerol trioctanoate, and glycerol triisopalmitate; hydrocarbons such as liquid paraffin, squalane, and pristane; higher fatty acids such as oleic acid, tall oil fatty acids, and isostearic acid; higher alcohols such as lauryl alcohol, oleyl alcohol, isostearyl alcohol, and octyldodecanol; silicone oils such as methylpolysiloxane, methylphenylpolysiloxane, methylhydrogenpolysiloxane, and decamethylpolysiloxane; ester oils such as isopropyl myristate, isopropyl palmitate, hexyl laurate, oleyl oleate, decyl oleate, octyldodecyl myristate, hexyldecyl dimethyloctanoate, diethyl phthalate, and dibutyl phthalate; and triacylglycerols such as tripalmitoylglycerol, 1-palmitoyl-2,3-oleoylglycerol, 1,3-oleoyl-2-palmitoylglycerol, 1-palmitoleoyl-2-stearoyl-3-inoleoylglycerol, 1-linoleoyl-2-palmitoleoyl-3-stearoylglycerol. Such fats and oils for use herein also include those obtained by subjecting the above fats and oils to a treatment such as hydrogenation or separation. The fats and oils may contain an unsaturated fatty acid, a side-chain fatty acid, a diglyceride, a monoglyceride, and other glyceride components, as long as the amount thereof is trivial.

Preferably, the permeation enhancer is squalane, glycerin, or a mixture thereof.

The emulsion component may comprise a citrus peel extract, for example lemon peel extract. For example, a commercially available lemon peel extract is HERBASOL citron from Cosmetochem.

Other citrus peel extracts include those with actives such as hesperidin methyl chalcone, dipeptide valyl-tryptophane (i.e., dipeptide-2 which comprises valine and tryptophan), and palmitoyl tetrapeptide-3 (which is the reaction product of palmitic acid and a synthetic peptide containing glycine, glutamine, proline, and arginine). Hesperidin, a bioflavonoid which can be found in citrus peel such as the peel of sweet oranges (Citrus aurantium var. sinensis), can be converted into hesperidin methyl chalcone by extracting hesperidin from its source and placing the extract into an alkaline solution. This converts hesperidin into hesperidin chalcone which can subsequently be methylated by any known methylation process to produce hesperidin methyl chalcone. Hesperidin methyl chalcone can strengthen capillary micro vessel barrier in or near skin area that has been contacted with a composition including this ingredient. Dipeptide valyl-tryptophane, which is commercially available under the trade name DIPEPTIDE VW through Sederma SAS (Cedex, France), can be used to mobilize fluid in skin tissue and drain the fluid from such tissue (which can reduce puffy eyes) when applied to the skin. Palmitoyl tetrapeptide-3, which is commercially available under the trade name N-PALMITOYL RIGIN through Sederma SAS (Cedex, France), can reduce local inflammation in skin tissue and restore skin firmness and elasticity when applied to skin. Further, a blend of these three ingredients is also commercially available under the trade name EYELISS through Dermaxime (Gauteng, South Africa).

Another example of a citrus peel extract includes Citrus grandis (Grapefruit) peel extract, which is believed to have has anti-hyaluronidase, anti-angiogenesis, anti- and inflammatory properties when applied to skin. This ingredient can be used as a soothing agent for acute or chronic inflammation and can help repair skin damaged from excessive UV exposure. An active ingredient in grapefruit extract is Apigenin. Grapefruit extract is commercially available under the trade name VIAPURE CITRUS through Actives International (Allendale, N.J.) and can also be isolated or purified from plants containing these extracts by standard isolation and purification techniques.

The emulsion component may further comprise any of a variety of additional cosmetically active agents. Examples of suitable additional active agents include: additional skin lightening agents, darkening agents, additional anti-aging agents, tropoelastin promoters, collagen promoters, anti-acne agents, shine control agents, anti-microbial agents such as anti-yeast agents, anti-fungal, and anti-bacterial agents, anti-inflammatory agents, anti-parasite agents, external analgesics, sunscreens, photoprotectors, antioxidants, keratolytic agents, detergents/surfactants, moisturizers, nutrients, vitamins, energy enhancers, anti-perspiration agents, astringents, deodorants, hair removers, hair growth enhancing agents, hair growth delaying agents, firming agents, hydration boosters, efficacy boosters, anti-callous agents, agents for skin conditioning, anti-cellulite agents, odor-control agents such as odor masking or pH-changing agents, and the like.

Examples of various suitable additional cosmetically acceptable actives include hydroxy acids, benzoyl peroxide, D-panthenol, UV filters such as but not limited to avobenzone (PARSOL 1789), bisdisulizole disodium (NEO HELIOPAN AP), diethylamino hydroxybenzoyl hexyl benzoate (UVINUL A Plus), ecamsule (MEXORYL SX), methyl anthranilate, 4-aminobenzoic acid (PABA), cinoxate, ethylhexyl triazone (UVINUL T 150), homosalate, 4-methylbenzylidene camphor (PARSOL 5000), octyl methoxycinnamate (Octinoxate), octyl salicylate (octisalate), padimate O (ESCALOL 507), phenylbenzimidazole sulfonic acid (Ensulizole), polysilicone-15 (PARSOL SLX), trolamine salicylate, bemotrizinol (TINOSORB S), benzophenones 1-12, dioxybenzone, drometrizole trisiloxane (MEXORYL XL), iscotrizinol (UVASORB HEB), octocrylene, oxybenzone (EUSOLEX 4360), sulisobenzone, bisoctrizole (TINOSORB M), titanium dioxide, zinc oxide, carotenoids, free radical scavengers, spin traps, retinoids and retinoid precursors such as retinol, retinoic acid and retinyl palmitate, ceramides, polyunsaturated fatty acids, essential fatty acids, enzymes, enzyme inhibitors, minerals, hormones such as estrogens, steroids such as hydrocortisone, 2-dimethylaminoethanol, copper salts such as copper chloride, peptides containing copper, coenzyme Q10, amino acids such a proline, vitamins, lactobionic acid, acetyl-coenzyme A, niacin, riboflavin, thiamin, ribose, electron transporters such as NADH and FADH2, and other botanical extracts such as oat, aloe vera, feverfew, soy, shiitake mushroom extracts, and derivatives and mixtures thereof.

Examples of suitable additional skin lightening active agents include, but are not limited to, tyrosinase inhibitors, melanin-degradation agents, melanosome transfer inhibiting agents including PAR-2 antagonists, exfolliants, sunscreens, retinoids, antioxidants, tranexamic acid, tranexamic acid cetyl ester hydrochloride, skin bleaching agents, linoleic acid, adenosine monophosphate disodium salt, chamomilla extract, allantoin, opacifiers, talcs and silicas, zinc salts, and the like, and other agents as described in Solano et al. Pigment Cell Res. 19 (550-571) and Ando et al. Int J Mol Sci 11 (2566-2575).

Examples of suitable tyrosinase inhibitors include but, are not limited to, derivatives of Vitamin C, Vitamin E and its derivatives, Kojic Acid, Arbutin, resorcinols, hydroquinone, flavones e.g. licorice flavanoids, licorice root extract, Mulberry root extract, Dioscorea Coposita root extract, Saxifraga extract and the like, Ellagic acid, Salicylates and derivatives, Glucosamine and derivatives, Fullerene, Hinokitiol, Dioic acid, Acetyl glucosamine, 5,5′-dipropyl-biphenyl-2,2′-diol (Magnolignan), 4-(4-hydroxyphenyl)-2-butanol (4-HPB), combinations of two or more thereof, and the like.

Examples of vitamin C derivatives include, but are not limited to, ascorbic acid and salts, Ascorbic Acid-2-Glucoside, sodium ascorbyl phosphate, magnesium ascorbyl phosphate, and natural extract enriched in vitamin C.

Examples of vitamin E derivatives include, but are not limited to, alpha-tocopherol, beta, tocopherol, gamma-tocopherol, delta-tocopherol, alpha-tocotrienol, beta-tocotrienol, gamma-tocotrienol, delta-tocotrienol and mixtures thereof, tocopherol acetate, tocopherol phosphate and natural extracts enriched in vitamin E derivatives.

Examples of resorcinol derivatives include, but are not limited to, resorcinol, 4-substituted resorcinols like 4-alkylresorcinols such as 4-butyresorcinol (rucinol), 4-hexylresorcinol (SYNOVEA HR, Sytheon), phenylethyl resorcinol (SYMWHITE, Symrise), 1-(2,4-dihydroxyphenyl)-3-(2,4-dimethoxy-3-methylphenyl)-Propane (nivitol, Unigen) and the like and natural extracts enriched in resorcinols.

Examples of salicylates include, but are not limited to, 4-methoxy potassium salicylate, salicylic acid, acetylsalicylic acid, 4-methoxysalicylic acid and their salts. In certain preferred embodiments, the tyrosinase inhibitors include a 4-substituted resorcinol, a vitamin C derivative, or a vitamin E derivative.

In more preferred embodiments, the tyrosinase inhibitor comprises phenylethyl resorcinol, 4-hexyl resorcinol, or ascorbyl-2-glucoside.

Examples of suitable melanin-degradation agents include, but are not limited to, peroxides and enzymes such as peroxidases and ligninases. In certain preferred embodiments, the melanin-inhibiting agents include a peroxide or a ligninase.

Examples of suitable melanosome transfer inhibiting agents including PAR-2 antagonists such as soy trypsin inhibitor or Bowman-Birk Inhibitor, Vitamin B3 and derivatives such as niacinamide, essential soy, whole soy, soy extract. In certain preferred embodiments, the melanosome transfer inhibiting agents includes a soy extract or niacinamide.

Examples of exfolliants include, but are not limited to, alpha-hydroxy acids such as lactic acid, glycolic acid, malic acid, tartaric acid, citric acid, or any combination of any of the foregoing, beta-hydroxy acids such as salicylic acid, polyhydroxy acids such as lactobionic acid and gluconic acid, and mechanical exfolliation such as microdermabrasion. In certain preferred embodiments, the exfolliant includes glycolic acid or salicylic acid.

Examples of retinoids include, but are not limited to, retinol (Vitamin A alcohol), retinal (Vitamin A aldehyde), retinyl acetate, retinyl propionate, retinyl linoleate, retinoic acid, retinyl palmitate, isotretinoin, tazarotene, bexarotene, adapalene, combinations of two or more thereof and the like. In certain preferred embodiments, the retinoid is selected from the group consisting of retinol, retinal, retinyl acetate, retinyl propionate, retinyl linoleate, and combinations of two or more thereof. In certain more preferred embodiments, the retinoid is retinol.

Examples of antioxidants include, but are not limited to, water-soluble antioxidants such as sulfhydryl compounds and their derivatives (e.g., sodium metabisulfite and N-acetyl-cysteine, glutathione), lipoic acid and dihydrolipoic acid, stilbenoids such as resveratrol and derivatives, lactoferrin, iron and copper chelators and ascorbic acid and ascorbic acid derivatives (e.g., ascobyl-2-glucoside, ascorbyl palmitate and ascorbyl polypeptide). Oil-soluble antioxidants suitable for use in the compositions of this invention include, but are not limited to, butylated hydroxytoluene, retinoids (e.g., retinol and retinyl palmitate), tocopherols (e.g., tocopherol acetate), tocotrienols, and ubiquinones. Natural extracts containing antioxidants suitable for use in the compositions of this invention, include, but not limited to, extracts containing flavonoids and isoflavonoids and their derivatives (e.g., genistein and diadzein), extracts containing resveratrol and the like. Examples of such natural extracts include grape seed, green tea, black tea, white tea, pine bark, feverfew, parthenolide-free feverfew, oat extracts, blackberry extract, cotinus extract, soy extract, pomelo extract, wheat germ extract, hesperedin, Grape extract, Portulaca extract, Licochalcone, chalcone, 2,2′-dihydroxy chalcone, Primula extract, propolis, and the like.

Particularly suitable substituted resorcinols include 4-hexyl resorcinol and 4-octylresorcinol, particularly 4-hexyl resorcinol. The structures of 4-hexylresorcinol and 4-octylresorcinol are shown below:

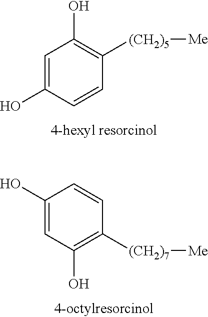

4-Hexyl resorcinol is commercially available as SYNOVEA HR from Sytheon of Lincoln Park, N.J. 4-Octylresorcinol is commercially available from City Chemical LLC of West Haven, Conn.

By “cotinus extract,” it is meant an extract of the leaves of Cotinus coggygria, such as a water extract thereof, available from Bilkokoop of Sofia, Bulgaria.

By “blackberry extract,” it is meant a blend of compounds isolated from the plant of the genus Rubus, and preferably Rubus fruticosus. In one embodiment, the compounds are isolated from the flowers of the plant. In a further embodiment, the compounds are isolated from dried flowers of the plant. Such compounds may be isolated from one or more part of the plant (e.g., the whole plant, flower, seed, root, rhizome, stem, fruit and/or leaf of the plant). In a preferred embodiment, the blackberry extract is a blackberry leaf extract.

One particularly suitable blackberry extract is produced by extracting the leaves of Rubus fruticosus with a mixture of water and ethanol compounded to an activity of about 5% to about 10%, with a maltodextrin matrix, commercially available from Symrise Inc. of Teterboro, N.J., and is sold under the name SYMMATRIX.

Extracts of “Phyllanthus niruri” may be harvested and used as the whole plant, or optionally one or more parts of the plant (e.g., flower, seed, root, rhizome, stem, fruit and/or leaf of the plant) may be used. The Phyllanthus niruri plant or parts thereof may be finely divided, such as by grinding or milling, to a powder. A suitable milled form of Phyllanthus niruri is commercially available from Raintree Nutrition, Inc., of Carson City, Nev. Preferably, a low molecular weight fraction of Phyllanthus niruri is used, for instance a fraction of Phyllanthus niruri substantially free of molecular species having a molecular weight of greater than about 100,000 daltons. Preferably, such low molecular weight fraction is water extractable from the Phyllanthus niruri plant.

A variety of other materials may also be present in the emulsion component, such as surfactants, chelating agents, emollients, humectants, conditioners, preservatives, opacifiers, fragrances and the like.

What is meant by an emollient is a compound that helps to maintain the soft, smooth, and pliable appearance of the skin (e.g., by remaining on the skin surface or in the stratum corneum to act as a lubricant). Examples of suitable emollients include those found in Chapter 35, pages 399-415 (Skin Feel Agents, by G Zocchi) in Handbook of Cosmetic Science and Technology (edited by A. Barel, M. Paye and H. Maibach, Published in 2001 by Marcel Dekker, Inc. New York, N.Y.), and include, but are not limited to, petrolatum, hexyldecyl stearate and plant, nut, and vegetable oils such as macadamia nut oil, rice bran oil, grape seed oil, palm oil, prim rose oil, hydrogenates peanut oil, and avocado oil.

What is meant by a humectant is a compound intended to increase the water content of the top layers of skin (e.g., hygroscopic compounds). Examples of suitable humectants include those found Chapter 35, pages 399-415 (Skin Feel Agents, by G

Zocchi) in Handbook of Cosmetic Science and Technology (edited by A. Barel, M. Paye and H. Maibach, Published in 2001 by Marcel Dekker, Inc. New York, N.Y.) and include, but are not limited to, glycerin, sorbitol or trehalose (e.g., α,α-trehalose, (β,β-trehalose, α,β-trehalose) or a salt or ester thereof (e.g., trehalose 6-phosphate).

What is meant by a surfactant is a surface-active agent intended to cleanse or emulsify. Examples of suitable surfactants include those found in Chapter 37, pages 431-450 (Classification of surfactants, by L. Oldenhove de Guertechin) in Handbook of Cosmetic Science and Technology (edited by A. Barel, M. Paye and H. Maibach, Published in 2001 by Marcel Dekker, Inc. New York, N.Y.) and include, but are not limited to anionic surfactants such as sulfates, cationic surfactants such as betaines, amphoteric surfactants such as sodium coco glycinate, noionic surfactants such as alkyl polyglucosides.

Examples of suitable chelating agents include those which are capable of protecting and preserving the compositions of this invention. Preferably, the chelating agent is ethylenediamine tetracetic acid (“EDTA”), and more preferably is tetrasodium EDTA, available commercially from Dow Chemical Company of Midland, Mich. under the tradename VERSENE 100XL.

Suitable preservatives include, for example, parabens, quaternary ammonium species, phenoxyethanol, benzoates, DMDM hydantoin, organic acids and are present in the composition in an amount, based upon the total weight of the emulsion component, from about 0 to about 1 percent or from about 0.05 percent to about 0.5 percent.

Any of a variety of commercially available pearlescent or opacifying agents are suitable for use in this invention. Examples of suitable pearlescent or opacifying agents include, but are not limited to, mono or diesters of (a) fatty acids having from about 16 to about 22 carbon atoms and (b) either ethylene or propylene glycol; mono or diesters of (a) fatty acids having from about 16 to about 22 carbon atoms (b) a polyalkylene glycol of the formula: HO-(JO)ₐ-H, wherein J is an alkylene group having from about 2 to about 3 carbon atoms; and a is 2 or 3;fatty alcohols containing from about 16 to about 22 carbon atoms; fatty esters of the formula: KCOOCH₂L, wherein K and L independently contain from about 15 to about 21 carbon atoms; inorganic solids insoluble in the emulsion component, and mixtures thereof.

Any fragrance compositions suitable for use on skin and desirable for a skin care composition may be used in the emulsion composition.

### Use

The composition may be applied to skin in need of treatment. For example, topical application may be made to any one or more of the skin of the face, lips, neck, chest, back, arms, axilla, hands, feet and/or legs in need of treatment for a condition described herein.

The composition may be applied directly from a package to the skin in need, by hand to the skin in need, or may be transferred from a substrate such as a wipe or mask, or a combination of two or more thereof. In other embodiments, the composition may be applied via a dropper, tube, roller, spray, patch or added to a bath or otherwise to water to be applied to the skin, and the like.

The composition is provided in a two-part package. The two-part package keeps the anhydrous component and the emulsion component separated until use.

The two-part package may comprise two containers, one containing the anhydrous component and one containing the emulsion component. The user may obtain the desired amount of anhydrous component and emulsion component from each, mix together, preferably until substantially homogeneous or homogeneous, and then preferably within 2 minutes, more preferably within 1 minute, of mixing, apply to the skin in need.

The two-part package may alternatively comprise two side-by-side tubes that mechanically dispense predetermined amounts, for example substantially equal amounts, of the two components at the same time.

A variety of two-part packages are known in the cosmetic and personal care art, and any such packages may be used.

### EXAMPLE 1

A two-part composition according to the invention was prepared as follows. An anhydrous component was made using powdered L-ascorbic acid dispersed in dimethicone/etearyl dimethicone crosspolymer as shown in Table 1.

TABLE 1

| | % w/w |
|---|---|
| Dimethicone/Cetearyl Dimethicone Crosspolymer | 80 |
| Ascorbic Acid | 20 |

Dimethicone/cetearyl dimethicone crosspolymer were added to a glass beaker and ascorbic acid was then added slowly thereto under stirring and mixed vigorously until uniform.

An oil-in-water emulsion component contained the ingredients shown in Table 2.

TABLE 2

| | % w/w |
|---|---|
| Water | qs to 100 |
| Citric Acid | 0.008 |
| Dissodium Phosphate | 0.015 |
| Glycerin | 12 |
| Distearyldimonium Chloride | 5 |
| Cetyl Alcohol | 2.5 |
| Dimethicone | 1.25 |
| Petrolatum | 4 |
| Isopropyl palmitate | 3 |
| Vitamin E | 0.1 |
| Benzyl Alcohol | 0.6 |
| Fragrance | 0.2 |

The oil-in-water emulsion component was prepared using the following procedure. In a primary glass beaker water, citric acid, glycerin and disodium phosphate were added and heated under stirring until 75-77° C. In a secondary glass beaker, distearyldimonium chloride, cetyl alcohol, dimethicone, petrolatum, and isopropyl palmitate were added and heated under stirring to 75-77° C. When both beakers reached 75-77° C., the contents of the secondary beaker were added to the primary beaker under stirring and the resulting mixture cooled to 35° C. Finally benzyl alcohol and fragrance were added and mixed until uniform.

The two components were blended in a 1:1 weight ratio. The anhydrous component contained 20% of L-ascorbic acid. When combined with the oil-in-water emulsion component, the final concentration of L-ascorbic acid was 10%.

### EXAMPLE 2

Another two-part composition according to the invention was prepared as follows. The anhydrous component contained the ingredients shown in Table 3.

TABLE 3

| | % w/w |
|---|---|
| Dimethicone/Cetearyl Dimethicone Crosspolymer | 60 |
| Ascorbic Acid | 32 |
| Caprylyl Methicone | 4 |
| Dimethicone | 4 |

In a glass beaker dimethicone/cetearyl dimethicone crosspolymer, caprylyl methicone and dimethicone were added and mixed until uniform. Ascorbic acid was then added slowly under stirring and mixed vigorously until uniform.

The oil-in-water emulsion component was made using the ingredients shown in Table 4.

TABLE 4

% w/w

Pre Mix

| | |
|---|---|
| Water (1st portion) | 5 |
| Glycerin (1st portion) | 6 |
| Panthenol | 0.5 |

Other ingredients

| | |
|---|---|
| Water | qs to 100 |
| Dicaprylyl Carbonate | 1 |
| Isononyl Isononanoate | 1 |
| Dimethicone | 0.5 |
| Squalane | 1 |
| Caprylyl Methicone | 3 |
| Phenoxyethanol | 0.5 |
| Glyceryl Stearate/PEG-100 Stearate | 1.5 |
| Dissodium EDTA | 0.1 |
| Cetearyl Olivate/Sorbitan Olivate | 1.5 |
| Cetyl palmitate | 2.0 |
| p-anisic acid | 0.3 |
| Cetyl Alcohol | 2.0 |
| Vitamin E acetate | 0.2 |
| Hydroxyethyl Acrylate/Sodium | 1 |
| Acryloyldimethyl Taurate | |
| Copolymer/Isohexadecane/Polysorbate 60 | |
| Polyacrylate-13/Polyisobutene/Polysorbate | 0.5 |
| 20 | |
| Glycerin(2nd portion) | 1 |
| Sodium Hydroxide | 0.12 |
| Fragrance | 0.25 |
| Aqua/Citrus Limon (Lemon) Peel Extract | 1.0 |
| Ethylhexylglycerin | 1.0 |
| Aluminum Starch Octenylsuccinate | 2.0 |
| Silica | 1.0 |

In a primary glass beaker a premix of water, glycerin and panthenol were added and mixed until the panthenol was completely solubilized. In second beaker, water, dissodium EDTA, glycerin (2nd portion), and sodium hydroxide were added and heated to 75-80° C. under stirring. In a third beaker dicaprylyl carbonate, isononyl isononanoate, dimethicone, squalane, caprylyl methicone, phenoxyethanol, glyceryl stearate/PEG-100 stearate, cetearyl olivate/sorbitan olivate, and cetyl palmitate were added and heated to 75-80° C. under stirring. When the second and third beakers reached 75-80° C., the contents of the third beaker were added to the second beaker under stirring and hydroxyethyl acrylate/sodium acryloyldimethyl taurate copolymer/isohexadecane/polysorbate 60, and polyacrylate-13/polyisobutene/polysorbate 20 were added and the mixture was homogenized for two minutes into an emulsion and then cooled to 35° C. The contents of the primary beaker were added to the emulsion and mixed until uniform. Finally fragrance, aqua/Citrus Limon (Lemon) Peel Extract, ethylhexylglycerin, aluminum starch octenylsuccinate and silica were added and mixed until uniform. The pH of the mixture was adjusted to 5.2-6.2 with citric acid.

The two components were blended in a 1:1 weight ratio.

### EXAMPLE 3

Twenty-five gram samples of the anhydrous component of Example 2 were subjected to stability testing as follows. The samples remained under 40° C.±75% relative humidity for three months during which time the ascorbic acid concentration was determined Analysis was carried out in a HPLC equipped with a UV detector. The data acquisition and processing were performed by the HPLC software. For all the samples, ascorbic acid analysis was performed in a Zorbax Carbohydrate part no. 840300-908, 5 μm, 4.6×250 mm, using as mobile phase a mixture of 35% of phosphate buffer (pH=2.5) and 65% of methanol for 10 minutes. The injection volume was 2 μL, flow rate was 1 mL/min and column temperature was set at 25° C. The chromatograms were processed at 245 nm.

The product was centrifuged and 300 μL of an internal standard (p-anisic acid) was added to the mixture. Afterward 10 mL of a solution of MeOH/water (80:20) and 3 mL of chloroform were added and the mixture was mixed in a vortex for one minute, followed by five minutes in an ultrasound bath. The sample was mixed in a vortex for another minute and then filtered in Millipore Membrane.

The results are shown in Table 5. The ascorbic acid concentration at initial time (day 0), 1 month and 3 months remained constant.

TABLE 5

| Sample | % Vitamin C Concentration |
|---|---|
| Initial (day 0) | 32.18 |
| 1 month | 32.52 |
| 3 months | 32.86 |
| Average | 32.52 |
| S.D. | 0.34 |
| RSD (%) | 1.0 |

### EXAMPLE 4

The ascorbic acid percutaneous permeation and cutaneous retention from: (1) a two-part composition according to the invention, and (2) an anhydrous ascorbic acid-containing component alone (comparative) were compared as follows.

The in vitro permeation of ascorbic acid from the compositions was evaluated using a Franz diffusion cell according to the OECD test guideline, Guidance Document for the Conduct of Skin Absorption Studies, Organization for Economic Cooperation and Development (OECD), Environment Directorate, OECD Environmental Health and Safety Publications, Series on Testing and Assessment No. 28, Paris, 2004.

The Franz diffusion cell (Hanson Research, Chatsworth, Calif.) had a static receptor solution reservoir with a side-arm sampling port. The receptor chamber had a volume of approximately 7 mL. The receptor liquid was PBS pH 7.4 with Cysteine 50 mM. The diffusion area was 2.0 cm² (round with 16 mm diameter).

Skin from pig ears was used. The skin samples had a maximum thickness of 0.5 mm. The skin samples were washed with a PBS buffer containing antibiotics and stored at −20° C. until their use. Half an hour before use the skin samples were removed from the freezer and kept at room temperature.

The skin samples were positioned in the Franz cell dermal side facing the receptor chamber and in contact with the receptor liquid for 30 minutes. The experiment was conducted at infinite dose. After the hydration time, a test composition was placed over the skin using a repipettor and spread using a capillary. For each test composition, five samples of receptor liquid were collected at 2 h, 4 h, 6 h and 8 h after application. Collected receptor liquid was analyzed by HPLC (HPLC Alliance from Waters® with a chromatographic column Luna C18 5 μm, 4.6 mm×250 mm and mobile phase with a flow of 1.0 mL/min.) for level of ascorbic acid.

The results are shown in Tables 6 (two part composition according to the invention) and 7 (comparative).

TABLE 6
Two Part Composition

| | Sample | Sample | Sample | Sample | Sample | | sd |
|---|---|---|---|---|---|---|---|
| Time | 1 | 2 | 3 | 4 | 5 | average | error |
| (h) | Vitamin C permeation (10⁻⁶ g/cm²) | | | | | | |
| 2 | 3.6 | 16.2 | 4.1 | 22.7 | 29.3 | 15.2 | 5.1 |
| 4 | 6.4 | 34.8 | 8.4 | 46.7 | 44.3 | 28.1 | 8.7 |
| 6 | 11.1 | 67.3 | 18.6 | 90.3 | 63.3 | 50.1 | 15.2 |
| 8 | 15.0 | 91.8 | 28.8 | 127.2 | 85.6 | 69.7 | 20.9 |

TABLE 7
Anhydrous Component Alone (Comparative)

| | Sample | Sample | Sample | Sample | | sd |
|---|---|---|---|---|---|---|
| Time | 1 | 2 | 3 | 4 | average | error |
| (h) | Vitamin C permeation (10⁻⁶ g/cm²) | | | | | |
| 2 | 2.8 | 1.4 | 3.7 | 1.8 | 2.4 | 0.5 |
| 4 | 4.8 | 3.0 | 5.2 | 3.5 | 4.1 | 0.5 |
| 6 | 8.4 | 5.6 | 8.4 | 8.2 | 7.6 | 0.7 |
| 8 | 11.2 | 7.9 | 10.6 | 12.4 | 10.5 | 0.9 |

The results show that the combination of the anhydrous component with the oil-in-water emulsion component provided superior permeation of Vitamin C though the skin when compared with treatment with only an anhydrous component (p<0.05 at all time-points using independent t-test). For example, at 8 h the vitamin C permeation was more than 6 times greater using the two part composition according to the invention (69.7×10⁻⁶ g/cm² versus 10.5×10⁻⁶ g/cm²).

The skin samples from the Franz diffusion cell were then subjected to “tape stripping” using Scotch 3M tape as follows. Sixteen strips were collected from each skin sample, and the first two strips were disposed of Each strip was taken after application at a constant pressure of 0.35 kgf/cm² for 15 seconds. After each strip the direction of the adhesive removal was modified by 45°.

The obtained tapes and skin samples were subjected to active extraction as follows.

Tubes containing 4 mL of PBS pH 7.4 with cysteine 50 mM were prepared. The edges of the tapes that had not been in contact with skin were removed. The remainders of the tapes were each put in a tube and submerged in the PBS/cysteine. Each tube was mixed for three minutes using a vortex mixer. Next, each tube was subjected to a sonication bath for 30 minutes. After sonication, the supernatant was removed and analyzed for ascorbic acid content using HPLC as described above.

The amount of ascorbic acid in the tapes, and therefore in the stratum corneum removed from the skin samples, is shown in Table 8.

TABLE 8
Retention of Vitamin C in Stratum Corneum (10⁻⁶ g/cm²)

| | | Anhydrous |
|---|---|---|
| | | component |
| Sample | Two Part Composition | (comparative) |
| 1 | 189.4 | 84.3 |
| 2 | 251.2 | 89.5 |
| 3 | 154.8 | 112.2 |
| 4 | 159.0 | 246.5 |
| 5 | 120.9 | 109.0 |
| average | 175.1 | 128.3 |
| sd error | 21.9 | 33.6 |

Each of the skin samples were then cut, fragmented and put in 50 mL flasks. Four mL of PBS pH 7.4 with cysteine 50 mM was added to each flask, and each mixture was homogenized using an ultra-turrax (speed of 25,000 rpm) until a homogeneous suspension was obtained. Each suspension was then homogenized using a vortex mixer for about one minute and left in ultrasonic bath for 30 minutes. After the sonication, the samples were centrifuged at 13,000 rpm and the supernatant was removed, filtered and analyzed for ascorbic acid content using HPLC as described above.

The amount of ascorbic acid remaining in the skin samples, and therefore in the dermis/epidermis, is shown in Table 9.

TABLE 9
Retention of Vitamin C in Derm-Epiderm (10⁻⁶ g/cm²)

| | | Anhydrous |
|---|---|---|
| | | component |
| Sample | Two Part Composition | (comparative) |
| 1 | 49.6 | — |
| 2 | 34.9 | 21.3 |
| 3 | 49.6 | 21.9 |
| 4 | 101.8 | 27.0 |
| 5 | 81.5 | 43.5 |
| average | 63.5 | 28.4 |
| sd error | 12.2 | 5.2 |

p<0.05 compared to the ascorbic acid in Derm/Epiderm from both treatments. Independent t-test was used.

We claim:

1. A two-part composition for topical application of ascorbic acid to skin comprising: a) an anhydrous component consisting essentially of greater than 15 weight % ascorbic acid suspended in a silicone vehicle; and b) an oil-in-water emulsion component comprising a permeation enhancer; wherein said anhydrous component and said oil-in-water emulsion component are separated until topically applied to said skin.

2. The composition of claim 1, wherein said silicone vehicle is a combination of a siloxane crosspolymer and a siloxane.

3. The composition of claim 1, wherein said oil-in-water emulsion component further comprises a citrus peel extract.

4. The composition of claim 1, wherein said permeation enhancer is selected from the group consisting of squalane, glycerin, and mixtures thereof.

5. A method of administering ascorbic acid to skin, which comprises in sequence: (1) mixing a) an anhydrous component consisting essentially of greater than 15 weight % ascorbic acid suspended in a silicone vehicle with b) an oil-in-water emulsion component comprising a permeation enhancer to form a mixture; and (2) topically applying said mixture to said skin within two minutes of said mixing.

6. The method of claim 5, wherein said silicone vehicle is a combination of a siloxane crosspolymer and a siloxane.

7. The method of claim 5, wherein said oil-in-water emulsion component further comprises a citrus peel extract.

8. The method of claim 5, wherein said permeation enhancer is selected from the group consisting of squalane, glycerin, and mixtures thereof.
